(19) United States
(12) Patent Application Publication
(10) Pub. No.: US 2021/0187235 A1
(43) Pub. Date: Jun. 24, 2021

(54) WEARABLE PERSONAL HUMIDIFIER

(71) Applicant: Sean Sweeney, Stillwater, NY (US)

(72) Inventor: Sean Sweeney, Stillwater, NY (US)

(21) Appl. No.: 16/720,357

(22) Filed: Dec. 19, 2019

(51) Int. Cl. A61M 16/16; B01F 3/04; A61M 16/06; A41D 20/00

(52) U.S. Cl. CPC A61M 16/16; B01F 3/04085; A61M 16/0644; A61M 2209/088; A41D 20/00; B01F 2215/0091; A41D 2400/32; A61M 16/0683

(57) ABSTRACT

Disclosed herein is a wearable personal, humidifier that includes a reservoir for holding water including an output port, a water delivery material including a hydrophilic porous or hydrophilic fibrous material, wherein the water delivery material is configured to make an airtight seal with the output port of the reservoir. The water delivery material includes a cavity having an opening to the output port of the reservoir. The output port is located on the bottom of the reservoir when the wearable personal humidifier is worn such that the flow of water from the reservoir to the water delivery material is self-regulated. As a user breathes, water from the water delivery material evaporates into the air consumed by the user and water from the reservoir automatically re-saturates the water delivery material.

### FIELD OF TECHNOLOGY

The following relates generally to humidification of air. More specifically, the following relates to wearable, personal humidifiers.

### BACKGROUND

Oftentimes, during an illness such as the common cold or strep throat one becomes acutely sensitive to the dryness of the air. The mucus membranes of the back of the throat become dry due to the flow of air of relatively low relative humidity over them during breathing. This is especially the case when breathing through the nose but can also occur when breathing through the mouth.

The situation is often worsened during sleeping when one does not periodically drink water to moisten a dry throat. Even when healthy, allergy sufferers can be more prone to irritation from air-borne allergens such as pollen when their nasal passageways are dry. This situation could be alleviated by humidifying the air of the room with a humidifying device for the entire room. Alternatively, a device for humidifying air on a personal level could be used, such as for sleep apnea whereby humid air of increased pressure is delivered through a mask or nasal tubing directly to the breath stream via a continuous positive airway pressure (CPAP) machine.

The first case, humidifying the whole room, can cause unpleasant sleeping conditions if the temperature of the room is high, as during summer if no air conditioning is used. Additionally, humidity can aid in the growth of dust mites and/or mold that can be bad for allergies. Moreover, condensation upon personal items and/or windows in the room due to excessive humidity can cause property damage.

The second case of personal humidifiers typically requires electrical power, can be relatively expensive (as in the case of CPAP machines), creates some noise, and is somewhat cumbersome to travel with.

As such, a device that addresses some or all of the above-mentioned needs would be well received in the art.

### SUMMARY

According to one embodiment, a wearable, personal humidifier comprises a sealable and impermeable reservoir for holding water, a water delivery material comprising a hydrophilic porous or fibrous material, wherein the water delivery material is configured to make an airtight seal with an output port of the reservoir, wherein the water delivery material includes a cavity having an opening to the output port of the reservoir, and wherein the output port is on the bottom of the reservoir such that the flow of water from the reservoir to the water delivery material is self-regulated.

According to another embodiment, a humidification device comprises a reservoir configured to hold water, a water delivery system integrally attached on the bottom of the reservoir; and an evaporation material located at the bottom of the water delivery system, wherein the evaporation material is at least one of a hydrophilic porous or fibrous material such that as water evaporates off the evaporation material, the evaporation material automatically pulls water from the reservoir through the water delivery system.

According to another embodiment, a personal humidification device comprises a water delivery tube having a first end and a second end, an impermeable container integrally attached to the first end of the water delivery tube, a water delivery material configured at the second end of the water delivery tube wherein the water delivery material makes an airtight seal with the second end of the water delivery tube, and a mask configured to be placed over the nose and/or mouth of a user, wherein the water delivery tube passes through the mask such that the first end of the water delivery tube is located outside of the mask and the second end of the water delivery tube passes into the inside of the mask, wherein the impermeable container is configured to hold water and the water delivery material is porous and hydrophilic such that as water evaporates off of the water delivery material, water inside the impermeable container is forced through the water delivery tube and on to the water delivery material.

### BRIEF DESCRIPTION OF THE DRAWINGS

The subject matter which is regarded as the invention is particularly pointed out and distinctly claimed in the claims at the conclusion of the specification. The foregoing and other features and advantages of the invention are apparent from the following detailed description taken in conjunction with the accompanying drawings in which:

FIG. 1 depicts a front view of a wearable, personal humidifier, according to embodiments of the present invention;

FIG. 2 depicts a magnified view of a water delivery material, according to embodiments of the present invention; and

FIG. 3 depicts a perspective view of a wearable, personal humidifier, according to embodiments of the present invention.

### DETAILED DESCRIPTION

A detailed description of the hereinafter described embodiments of the disclosed apparatus and method are presented herein by way of exemplification and not limitation with reference to the Figures.

The average person, while sedentary, breathes approximately 7 liters of air per minute. If this air starts at a temperature of 22.2 degrees Celsius (72 degrees Fahrenheit), and a relative humidity of 30%, then to achieve a relative humidity of 90% at this temperature, 66 milligrams of water per minute would have to be evaporated into the breath stream while breathing. Over the course of 8 hours of sleeping, this corresponds to 31.7 grams (1.11 ounces) of water. Water on the order of this amount could readily be carried in a reservoir held on one's head or face. Thus, the air one breathes could be humidified for an entire night's sleep using a device attached to one's head or face without the need for refilling with water during the night.

Embodiments of the invention disclosed herein may provide a number of advantages. For example, in some embodiments, the device disclosed herein allows for personal humidification of air such that only the air close to the mouth or nose of a user is humidified. In this way, the temperature and humidity of entire rooms or larger areas are not modified in any significant way. Additionally, in some embodiments, the device allows for automatic refilling with water of the area which is humidifying the air. Conventional humidifiers require a pump or some other monitoring of the water levels. In contrast, the humidifier disclosed herein automatically pulls water in to the water delivery area as water evaporates off of the water delivery material. For example, the device disclosed herein may enable a silent (other than breathing noises) supply of moisture to be delivered to the breathing path of a person for several hours, as during sleeping. Additionally, in some embodiments the device may not cause a significant increase in humidity in the surrounding room. Moreover, in some embodiments, the device may not require batteries or electric power, and may have a high level of portability.

The device disclosed herein may be comprised of a reservoir for holding water, a water delivery area, comprised of a water delivery material, for humidifying the air that a user breathes. There may be a seal between the water delivery material and a water delivery system of the device that extends from the reservoir during operation of the device. The reservoir, or container for holding water, can be constructed of a variety of water-impermeable materials, although a polymeric material is preferable because of its light-weight and relatively low cost. The reservoir may be any shape or size suitable for holding water. For example, the reservoir may be cube shaped, cylindrical, spherical, rectangular, triangular, or the like.

In one embodiment of the device, the water delivery system is a tube that the water delivery area seals to, and which allows water to flow from the reservoir to the water delivery area and air to flow from the water delivery area to the top of the reservoir. The water delivery area may be in or near the breathing path of a user and the water delivery material held in place by a hydrophobic material to avoid liquid water collection at the interface between the water delivery material and the hydrophobic material. The water delivery area delivers water vapor to the breath stream of a person.

The water delivery material may be a hydrophilic porous material capable of retaining liquid water in its pores through capillary force against the force of gravity and inertia (as during movement of the device). The water delivery material may also be referred to as an evaporation material. The water delivery material would preferably be of a shape for increased surface area through which to pass water vapor into the breath stream, such as a zig zag, or a star-shaped tube (described in more detail below). Water lost through evaporation from the water delivery material is replaced by water from the reservoir which flows down to the water delivery area through the water delivery system while air bubbles rise from the interface of the water delivery material and the water in the water delivery system. The air bubbles at the interface of the water delivery material and the water in the water delivery system form due to air flow/diffusion from the evaporation surface of the water delivery area through the pores of the water delivery area, while water concurrently flows through pores in the other direction due to capillary pressure-driven transport. This water replenishment process is self-regulating from a balance of gravity and capillary pressure acting against a partial vacuum that forms above the water in the reservoir, and this process does not result in water dripping off of the water delivery area. As the reservoir and the interface between the water delivery material and the water delivery system can both be sealed, as air bubbles form in the water delivery material, the only path for them to take is up the water delivery system and into the reservoir. The added volume of air to the reservoir results in water being displaced. This displaced water pushes water down the water delivery system on to the water delivery material, thereby replacing the air bubble which detached and traveled into the reservoir.

The water delivery material may contain a cavity that is open to the water delivery system and that allows water to more easily flow to the sections of the water delivery material that are furthest from the water delivery system, thus maintaining moistness of all parts of the water delivery material under high evaporation rate conditions (low relative humidities).

Another advantage of the device disclosed herein over previous devices is that its construction creates a self-regulation of the flowrate of water coming from the reservoir and evaporating into the breath stream, and no external power is needed to maintain this flowrate.

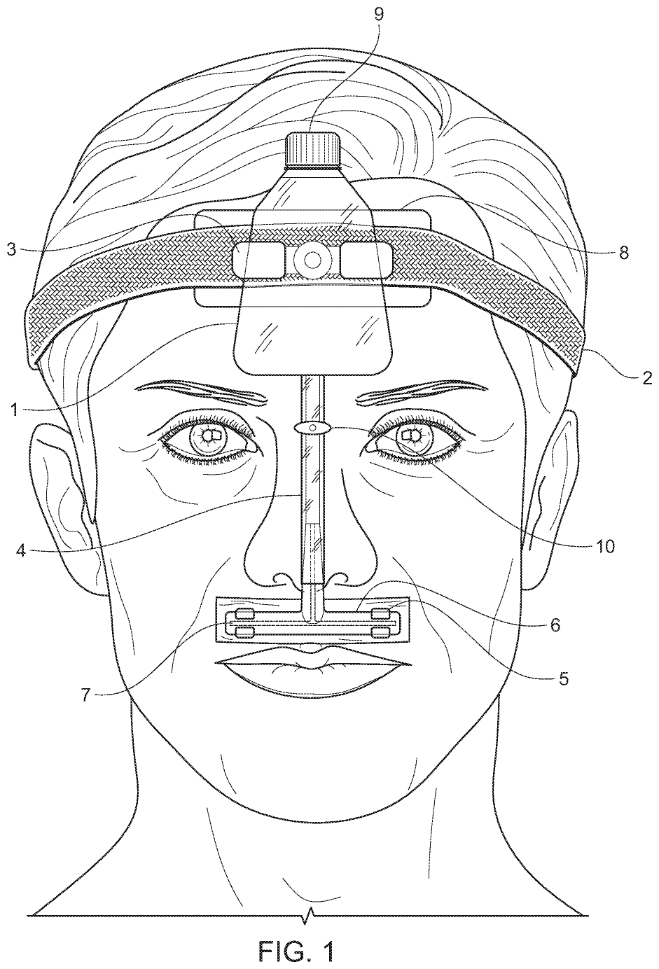

Referring to the drawings, FIG. 1 depicts a front view of a wearable, personal humidifier, according to embodiments of the present invention. The reservoir 1 may have a rigid, impermeable body, such as a plastic container that gets filled with water. For example, the reservoir 1 may be impermeable to both air and water. The reservoir 1 may be a container, a receptacle, a box, a bucket, a jug, or any other vessel capable of holding water. The reservoir 1 may be sealed. This means that, if an opening exists to add water to the reservoir 1, this opening is capable of being sealed, such as by a screw cap or a top or any other top capable of forming an airtight seal. The reservoir 1 can be constructed of a variety of water-impermeable, air tight materials, such as a relatively stiff polymeric material. The reservoir 1 may have a capacity in the range of about 0.02 fluid ounces (0.59 mL) to about 12 fluid ounces (355 mL), or in other embodiments, in the range of about 0.1 fluid ounces (2.96 mL) to about 1.5 fluid ounces (44.4 mL).

The reservoir 1 may be attached to a person's forehead through use of an attachment band 2. The attachment band 2 may utilize a holding clip 3 configured on the humidification device. The attachment band 2 may be an elastic band, a string, an adjustable headband, a wearable headgear (such as a hat, helmet, hood, or the like), an adhesive, a ratchetting assembly, a padded/coated wire or any other attachment device for fixing the device to the head of a user. For example, as shown in FIG. 1, the attachment band 2 may include an elastic band that threads through a holding clip 3 attached to the reservoir 1. A padded shim 8 can be inserted between the holding clip 3 and a user's head.

Water delivery system 4 connects the reservoir 1 and the water delivery area 6. The water delivery system 4 may be a tube, an opening at the bottom of the reservoir 1, an outlet port, a pipe, a hole in the reservoir 1, or any other means for allowing water to exit from the bottom of the reservoir 1. For example, as shown in FIG. 1, the water delivery system 4 may include a tube, tracing a path over a user's nose to reach the upper lip where air that is breathed in through their nostrils passes over the water delivery material 6. The water delivery system 4 may have a diameter from about 0.1 cm to about 5 cm, or in other embodiments, from about 0.4 cm to about 2 cm.

The reservoir 1 may be attached to a user using the attachment band 2. The reservoir 1 could also be carried on the head of a user via a temporary adhesive. The reservoir 1 could also be held by a pocket in a hat or other article worn on the head of a user. In the example embodiment shown in FIG. 1, with the reservoir 1 being worn attached to the head of a user, the water delivery system 4 could be routed over the nose.

The water delivery material 6 is the area which provides water to the air consumed by a user. The water delivery material 6 may be located proximate to the nose and/or mouth of a user such that as a user breathes, the air that enters the nose and/or mouth passes over the water delivery material 6. The area near a user's nose and/or mouth may include the water delivery material 6 and holder 5.

The water delivery material 6 may make an air-tight seal with the water delivery system 4. For example, if the water delivery system 4 is tubular in nature, the water delivery material 6 may make a seal with the end of the water delivery system 4 located proximate to the water delivery material 6 such that water is able to flow into the water delivery material 6, but air is not allowed to enter or escape at the point where the water delivery material 6 and the water delivery system 4 meet, at least not without traveling through the pores of the water delivery material 6. In other words, at the point where the water delivery material 6 and the water delivery system 4 meet, the only path for air outside of the pores of the water delivery material 6 to travel would be up the water delivery system 4 and into the reservoir 1. In another embodiment, a section of the water delivery material 6 may insert a short distance into the water delivery system 4 to form a seal to prevent air from getting in and water from dripping out of the water delivery system 4 (i.e., all water and air is contained within the device and water is allowed to travel from the reservoir 1 to the water delivery material 6 and air from the water delivery material 6 to the reservoir 1, but neither is allowed to exit the device at this seal).

The water delivery material 6 may be made of a compressible, hydrophilic, porous material such as a polyvinyl alcohol (PVA) sponge material, a natural sponge material, a polyester sponge material, or a cellulose sponge material. Alternatively, the water delivery material 6 may be made of a relatively incompressible material such as a porous ceramic. The water delivery area may be a number of different shapes. For example, the water delivery material 6 may be rod shaped, flat, conformal to the lip of a person, in a zig-zag shape, a T-shape, a tube, a star-shaped tube, a spiral, a honeycomb, a trellis pattern or any other shape for increased surface area for moisture delivery into the breathing path of a user.

The water delivery material 6 may include a cavity 7. The cavity 7 may be a hole, a cavity, a groove, a notch, a recess, a depression, a cave, or the like. In some embodiments, the cavity 7 may be open to the water delivery system 4 and runs along the majority of the water delivery material 6's length. The cavity 7 may enable faster water replenishment from the reservoir 1.

The water delivery material 6 is held in place with a holder 5. The holder 5 may be a hydrophobic material. As shown in FIG. 1, the holder 5 may be a plastic above-lip holder stuck onto a person's top lip area using an adhesive backing. The area which contacts the lip of a user may be a soft plastic or rubber material. Additionally, the holder may be configured to make a seal with the nostrils of a user.

The water delivery system 4 may contain an on-off valve 10. The reservoir 1 may be filled with water through the hole of a fill cap 9 while the valve 10 is closed. The fill cap 9 may seal the reservoir 1 such that no water can exit. The fill cap 9 may be a screw cap, a snap cap, a valve, or the like. In other embodiments, the reservoir 1 may have no additional openings and no cap 9 is used. In such an embodiment, the reservoir 1 may be filled through the water delivery system 4.

The water delivery material 6 may be pre-saturated with water before use. The valve 10 may be closed and the fill cap 9 removed. Water may then be poured into the reservoir 1 through the fill cap 9 hole. The fill cap 9 is replaced on the reservoir 1 to make an airtight seal, and the valve 10 is opened. The device may then be attached to a user's head using the attachment band 2. A padded shim 8 can be inserted between the holding clip 3 and a person's forehead to maintain the reservoir 1 above the water delivery area 6 when a person is laying down.

When used in a vertical position such as during standing, walking, and sitting, the reservoir 1 is maintained above the water delivery material 6 with or without the padded shim 8. The water delivery material 6 is placed within the hydrophobic plastic holder 5 and the holder 5 is placed on a person's lip, such as by using an adhesive backing. The holder 5 may be hydrophobic to discourage liquid water from accumulating at the water delivery area interface with the holder 5 due to capillary pressure. Herein when the bottom of the reservoir 1 is referred to, the bottom should be understood to be the bottom of the reservoir 1 when the device is being worn in a vertical position, as shown in FIG. 1.

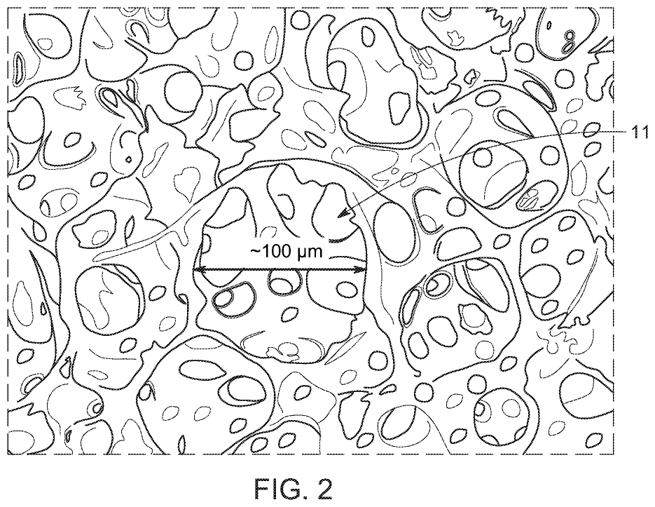

Embodiments of the invention described herein may be self-regulating, automatically refillable, or the like. This refers to the process described in this paragraph. During normal breathing, liquid water will evaporate from the water delivery material 6 as the breath passes over it and become entrained in the breath stream as added humidity. As water evaporates from the water delivery material 6, air diffuses/flows on a microscopic level through the pores 11 (shown schematically in FIG. 2 and described in more detail below) of the water delivery material 6 to reach the cavity 7 on the inside of the water delivery material 6. With further evaporation, air will continue to collect in the cavity 7 to form an air bubble of sufficient size to detach from inside the water delivery material 6 and rise up the water delivery system 4 to reach the reservoir 1 as liquid water concurrently drains down from the reservoir 1 to fill in the volume where the bubble detached at the interface of the water delivery material 6 and the water in the cavity 7 of the water delivery material 6. As the reservoir 1 is sealed, the added air volume to the reservoir 1 will automatically allow a corresponding volume of water to travel down the water delivery system 4. In other words, a combination of gravity and capillary pressure from the pores in the water delivery material 6 allow the water to travel from the reservoir 1 to the water delivery material 6. Air suction (lower air pressure than ambient) above the water prevents additional water or excess water from escaping the reservoir prior to the air bubble detaching and traveling to the reservoir. The air bubble rising decreases the air suction that restrains gravity and capillary pressure from forcing the water down to the water delivery material 6. Thus, water from the reservoir 1 replenishes the water that has evaporated into the breath stream from the water delivery material 6, and this maintains the water delivery material 6 nearly saturated as the breath stream is humidified. Thus, the entire system is self-regulating or automatic.

Moving to FIG. 2, a magnified view of water delivery material 6, according to embodiments of the present invention, is depicted. The water delivery material 6 may include pores 11 of non-monomodal size, i.e., having a bimodal, trimodal, or broad distribution of pore sizes to establish significant capillary pressure to drive water flow by containing smaller pores, while also reducing the resistance to water flow by containing larger pores. Pores 11 in the hydrophilic material of the water delivery material 6 have a broad range of sizes, with an average diameter, for instance, of 40 microns. Pore sizes could range from about a micron up to 300 microns or more. For example, average pore size may range from 10 to 600 microns. In other embodiments, the average pore size may range from 30 to 300 microns. This pore structure provides higher infiltration rates for faster replenishment of water from the reservoir 1 to the water delivery material 6 as water evaporates from the water delivery material 6 surface into the breath stream.

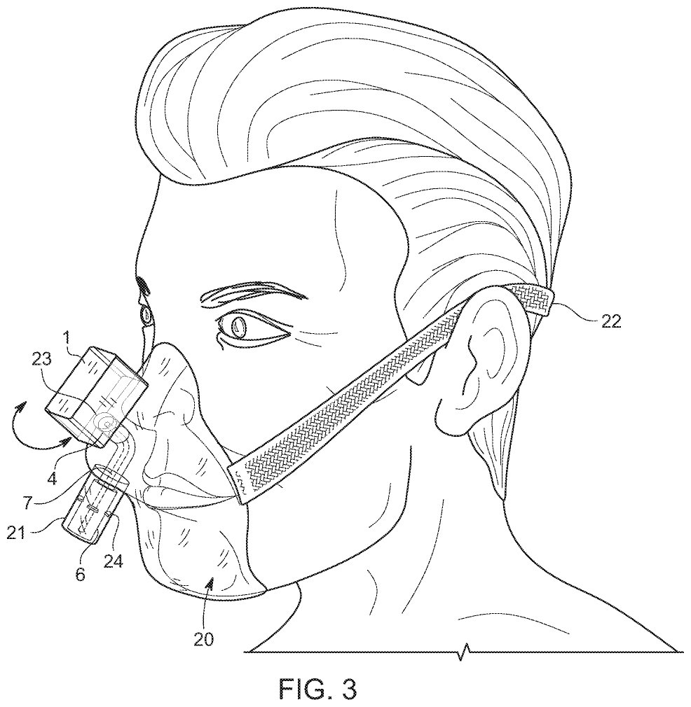

Turning to FIG. 3, a perspective view of a wearable, personal humidifier, according to embodiments of the present invention is depicted. In the example embodiment depicted in FIG. 3, the water delivery material 6 may held by centering supports 24 inside a breather tube 21 of a mask 20. The mask 20 may be shaped to cover the nose and mouth of a user. The mask 20 may be an impermeable mask made from a flexible material. The mask 20 should be flexible enough to make a seal with the face of a user. For example, the mask 20 may be made of vinyl or ethylene-vinyl acetate (EVA). The breather tube 21 may be made of a material that is stiffer than the material of the mask 20. For example, the breather tube 21 may be made from polypropylene, high-density polyethylene (HDPE), polyethylene terephthalate (PET), or any other suitable polymer. The breather tube 21 may be a number of suitable shapes and/or sizes. For example, the breather tube 21 may be cylindrical, spherical, rectangular, cubed, or the like.

The reservoir 1 may be held onto the mask 20 by the water delivery system 4. The water delivery system 4 may pass through a mask hole 23 in the mask 20. One end of the water delivery system 4 is attached to the reservoir 1 and the other end has a section of the water delivery material 6 inserted into it, making a seal. The mask 20 may make a seal around the nose and mouth area of a user and may be held in place through use of an attachment band 22 around the head of a user. The attachment band 22 may be similar to the attachment band 2, shown in FIG. 1.

The water delivery material 6 may held in place inside the breather tube 21 by hydrophobic centering supports 24. A cavity 7 may run through the majority of the length of the water delivery material 6 to enable faster water replenishment from the reservoir 1.

The water delivery material 6 may be removed from the water delivery system 4 and pre-saturated with water as it's held in the hydrophobic centering supports 24 in the breather tube 21 of the nose and mouth mask 20. The centering supports 24 may be hydrophobic to discourage liquid water from accumulating at the water delivery area interface with the centering supports 24 due to capillary pressure.

With the reservoir 1 upside down, water may be poured into the reservoir 1 through the water delivery system 4. The water delivery system 4 may be integrally attached to the reservoir 1. That is, the water delivery system 4 may be permanently attached to the reservoir 1. As described above, the reservoir 1 may also have a hole and cap located in the top of the reservoir 1 for adding water.

A section of the water delivery material 6 may be placed into the water delivery system 4 (such as a tube or pipe), and the device turned right side up. The reservoir 1 is rotated around the same axis as the mask hole 23 to get the reservoir 1 into the position corresponding to the orientation in which the device is to be used (such as in a vertical position, as during standing, walking, and sitting, or in a horizontal position, as during sleeping on one's back, or in a sideways horizontal position, as during sleeping on one's side).

The mask 20 may be placed on a user's head and held in place with the attachment band 22 (such as an elastic band or the like, as described above) wrapping around their head. When a breath is taken, the air is humidified due to the air passing through the breather tube 21 near the water delivery material 6 where water vapor is continuously evaporating from the porous, hydrophilic water delivery material 6. As water leaves the water delivery material 6 via evaporation, more water comes into the water delivery material 6 from the reservoir 1 through the water delivery system 4, thus keeping the water delivery material 6 moist, in a similar fashion to the self-regulating feature described above.

The device disclosed herein provides a means for humidifying the air one breathes on a personal basis with a self-regulating, non-electrically powered control of the flow of water from a reservoir to the breathing path.

Elements of the embodiments have been introduced with either the articles “a” or “an.” The articles are intended to mean that there are one or more of the elements. The terms “including” and “having” and their derivatives are intended to be inclusive such that there may be additional elements other than the elements listed. The conjunction “or” when used with a list of at least two terms is intended to mean any term or combination of terms. The terms “first” and “second” are used to distinguish elements and are not used to denote a particular order.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims. Moreover, it should be understood that the present invention may include any combination of the components, hierarchy and methodology described herein.

1. A wearable personal humidifier comprising:
a reservoir for holding water including an output port,
a water delivery material including a hydrophilic porous or hydrophilic fibrous material, wherein the water delivery material is configured to make an airtight seal with the output port of the reservoir,
wherein the water delivery material includes a cavity having an opening to the output port of the reservoir, and
wherein the output port is on the bottom of the reservoir when the wearable personal humidifier is worn such that the flow of water from the reservoir to the water delivery material is self-regulated.

2. The wearable personal humidifier of claim 1, wherein the output port of the reservoir is at least one of a hole in the bottom of the reservoir or a tube extending from the bottom of the reservoir.

3. The wearable personal humidifier of claim 1, wherein the output port of the reservoir has a diameter between 0.1 cm and 5 cm.

4. The wearable personal humidifier of claim 1 wherein the seal between the water delivery material and the output port of the reservoir is made by the water delivery material directly contacting and covering the entire output port.

5. The wearable personal humidifier of claim 1, wherein the water delivery material is comprised of at least one section that is rod shaped, flat, conformal to the lip of a person, configured in a zig-zag, T-shape, tube, star-shaped tube, spiral, honeycomb, trellis pattern or other shape for increased surface area for moisture delivery.

6. The wearable personal humidifier of claim 1, wherein said water delivery material is held in place by a hydrophobic material.

7. The wearable personal humidifier of claim 1, wherein the water delivery material comprises pores having an average diameter between 30 and 300 microns.

8. A humidification device comprising:
a reservoir configured to hold water;
a water delivery system integrally attached on the bottom of the reservoir when the humidification device is worn; and
an evaporation material located at the bottom of the water delivery system when the humidification device is worn;
wherein the evaporation material is at least one of a hydrophilic porous or fibrous material such that as water evaporates off the evaporation material the evaporation material automatically pulls water from the reservoir through the water delivery system.

9. The humidification device of claim 8, wherein the evaporation material forms an airtight seal with the water delivery system.

10. The humidification device of claim 8, wherein the water delivery system is at least one of an opening in the reservoir or a tube extending from the bottom of the reservoir.

11. The humidification device of claim 8, further comprising a mask configured to be placed over the mouth and nose of a user wherein the evaporation material is located at least partially inside the mask.

12. The humidification device of claim 8, further comprising an adjustable attachment system for fixing the humidification device on the head of a user, the attachment system having a body with a length.

13. The humidification device of claim 12, wherein the adjustable attachment system comprises at least one of:
a holding clip configured on the humidification device;
an elastic band;
a string;
an adjustable headband;
a wearable headgear; and
an adhesive.

14. A personal humidification device comprising:
a water delivery tube having a length and a first end and a second end;
a container integrally attached to the first end of the water delivery tube;
a water delivery material configured at the second end of the water delivery tube wherein the water delivery material makes an airtight seal with the second end of the water delivery tube; and
a mask configured to be placed over the nose and/or mouth of a user, wherein the water delivery tube passes through the mask such that the first end of the water delivery tube is located outside of the mask and the second end of the water delivery tube passes into the inside of the mask;
wherein the impermeable container is configured to hold water and the water delivery material is porous and hydrophilic such that as water evaporates off of the water delivery material, water inside the container is forced through the water delivery tube and on to the water delivery material.

15. The personal humidification device of claim 14, further comprising an attachment band for attaching the personal humidification device to the head of a user.

16. The personal humidification device of claim 14, wherein the water delivery material includes a cavity located at least partially at the second end of the water delivery tube.

17. The personal humidification device of claim 14, further comprising a breather tube, wherein the second end of the water delivery tube passes into the breather tube and the breather tube extends at least partially into the inside of the mask.

18. The personal humidification device of claim 17, wherein the water delivery material is configured inside the breather tube and is held in place by at least one hydrophobic support.

19. The personal humidification device of claim 14, wherein the container is constructed of a polymeric material.

20. The personal humidification device of claim 14, wherein the water delivery material is placed at least partially inside the second end of the water delivery tube to make the airtight seal.
